(19) United States
(12) Patent Application Publication
(10) Pub. No.: US 2008/0269479 A1
(43) Pub. Date: Oct. 30, 2008

(54) Process for Isolation of Macrolide Compounds

(75) Inventors: Martin Buchta, Ludgerovice (CZ); Ladislav Cvak, Opava (CZ); Josef Satke, Opava (CZ)

Correspondence Address: LERNER, DAVID, LITTENBERG,;KRUMHOLZ & MENTLIK, 600 SOUTH AVENUE WEST, WESTFIELD, NJ, 07090, US

(73) Assignee: IVAX Pharmaceuticals s.r.o., Opava 9 (CZ)

(21) Appl. No.: 11/662,234

(22) PCT Filed: Sep. 9, 2005
(86) PCT No.: PCT/US05/32249

§ 371 Date: Oct. 30, 2007

Related U.S. Application Data
(60) Provisional application No. 60/608,752, filed on Sep. 10, 2004.

(51) Int. Cl. C07D 273/01

(52) U.S. Cl. 540/456

(57) ABSTRACT

Although macrolide compounds are insoluble in water, surprisingly high part of the product was found in the liquid phase of the fermentation broth. Therefore, processing the entire fermentation broth, i.e. the suspension obtained by cultivation of a microorganism producing the required macrolide compound, is highly advisable. This invention teaches a method of processing the entire fermentation broth using cheap and environmentally acceptable solvents.

### FIELD OF THE INVENTION

The present invention relates to a process for isolation macrolide compounds, namely tacrolimus or sirolimus or their naturally occurring derivatives and analogues from fermentation broth.

### BACKGROUND OF THE INVENTION

Macrolide compounds or macrolides are multi-membered lactone rings. Erythromycin used as antibiotic is a well known example of such macrolide. Other macrolides such as tacrolimus and sirolimus are often used as immunosuppressants.

Tacrolimus, a macrolide with selective inhibitory effect on T-lymphocytes, was first described in U.S. Pat. No. 4,894,366 and European Patent EP 184,162. Tacrolimus was also described in scientific papers: H. Tanaka et al. J. Am. Chem. Soc. 1987, 109, 5031-5033 and T. Kino et al. J. Antibiot. 1987, 40, 1249-1255.

Sirolimus, also known as rapamycin, was first described in U.S. Pat. No. 3,929,992. Sirolimus was also described in scientific papers: C. Vezina et al. J. Antibiot. 1975, 28, 721-726, S. N. Sehgal et al. J. Antibiot. 1975, 28, 727-731.

Ascomycin, a macrolide, is a natural analogue of tacrolimus. Ascomycin is described in following papers: H. Hatanaka et al. J. Antibiot. 1988, 41, 1592-1599, M. Morisaki at al. J. Antibiot. 1992, 45, 126-132. Other natural derivatives and analogues of tacrolimus were described in patents EP 358,508 and GB 2,269,172.

Preferred process for tacrolimus and sirolimus preparation is fermentation, although total synthesis of both compounds has also been described (EP 378,318 and K. C. Nicolaou et al. J. Am. Chem. Soc. 1993, 115, 4419).

Isolation of both tacrolimus and sirolimus from fermentation broth is difficult due to their low concentration of in these macrolides biomass and due to fact, that the macrolides are present in both the solid phase (mycelium) and liquid phase (filtered fermentation broth). The process for economical isolation of a macrolide compound therefore requires (1) a separation of mycelium and (2) separation processing of both the mycelium and the filtered fermentation broth as described e.g. in T. Kino et al. J. Antibiot. 1987, 40, 1249-1255. Another possibility is described in patent application WO 03/68 980, claiming direct extraction of the fermentation broth with hydrophobic organic solvents.

### SUMMARY OF THE INVENTION

The process according to the invention makes possible processing of a whole fermentation broth. The extraction of a macrolide compound from the mycelium is accomplished by addition of a suitable water-miscible organic solvent to the whole broth. The macrolide compound is thereby transferred into a liquid phase. The extracted mycelium is then separated. The liquid phase (the aqueous extract) is further processed by extraction with a suitable water non-miscible solvent to obtain an organic extract. The organic extract is then partially evaporated and the residue is transferred into toluene to obtain a toluene concentrate. The toluene solution is further purified by chromatography on silica gel using toluene that has been polarized with acetone as a mobile phase. The fractions containing the macrolide compound are then concentrated and the residue is crystallized from a suitable solvent to obtain a desired macrolide compound.

In another embodiment of the process, the aqueous extract is not separated from the mycelium before subjecting to the treatment with a water non miscible solvent. The water non miscible solvent can be added directly to the suspension of mycelium in aqueous extract and the organic extract can be then separated from the three phase system.

### DETAILED DESCRIPTION OF THE INVENTION

Adding a suitable water-miscible organic solvent to the whole fermentation broth extracts macrolide compounds into the liquid phase. Such a water-miscible solvent can reduce co-extraction of aliphatic alcohols or ketones. Preferable solvents are acetone, 2-propanol and

1-propanol. Ethanol can be used for extracting macrolide compounds but it is less convenient than acetone and/or 2-propanol as ethanol can react with an isolated macrolide compound. The aqueous extract obtained by adding a water-miscible organic solvent to the whole fermentation broth can be separated from the extracted mycelium by filtration or by sedimentation, preferably by centrifugal separation. A clear aqueous extract will be obtained that can further processed without any evaporation. The aqueous extract can also be processed without separation of the solid phase.

Further processing of the aqueous extract, whether the mycelium is separated or not, comprises adding a water non miscible solvent to the aqueous extract and mixing the two or three phase system. Thereby, the macrolide compound is extracted to the organic phase, while most ballast components stay in the water phase. The water non-miscible solvent can be any organic water non-miscible solvent with exception of aliphatic hydrocarbons. Preferred solvents are toluene, xylene, dichloromethane, dichloroethane, tert-butyl methyl ether and isobutyl ketone. This invention discloses purification of a macrolide compound and concentration of the product, because only a very small amount of the water non-miscible solvent can be added to the aqueous extract to transfer the macrolide compound to the organic phase quantitatively, as demonstrated in the examples. Toluene is the preferred solvent because simple recovery of the used solvents due to substantial difference of boiling points of toluene and acetone or 2-propanol.

After the macrolide is extracted into the organic phase the separated organic phase is then concentrated under vacuum. The obtained concentrate is further purified by chromatography on silica gel using toluene stepwise polarized with acetone. The concentrate obtained by evaporation of the organic extract can be directly loaded to the chromatographic column. The final operation of the process according to the invention is crystallization of the chromatographic fractions containing the required macrolide compound from suitable solvents as described in the examples.

### EXAMPLES

### Example 1

10 liter of whole fermentation broth obtained by submerged cultivation of

Streptomyces sp. producing tacrolimus was diluted with 10 liter acetone and the suspension was stirred for 4 hours. Solid phase was separated by filtration and the filtrate was extracted two times with 1000 ml toluene. Toluene extracts were combined and toluene was evaporated under reduced pressure to form a concentrate of the volume about 100 ml. This concentrate was loaded to a chromatographic column filled with 100 g silica gel (Lichroprep Merck 60, 63-200 μm). The column was washed first with toluene (about 300 ml) and then with a mixture of toluene and 5 to 30% (v/v) acetone. The fractions containing tacrolimus (TLC monitoring) were combined and evaporated to dryness to produce a residue. The residue (3.7 g) was dissolved in 2-propanol (10 ml) and 20 ml water and 30 ml hexane was added to the solution. Crystallization of tacrolimus was accomplished by cooling the solution in a refrigerator (about +2° C.). Crystalline tacrolimus was separated by filtration. 1.4 g of crystalline tacrolimus was obtained.

### Example 2

10 liter of whole fermentation broth obtained by submerged cultivation of Streptomyces sp. producing sirolimus was diluted with 10 liter 2-propanol to form a suspension. The suspension was stirred for 4 hours. Solid particles were separated by filtration and the filtrate was extracted three times with 1000 ml toluene. The toluene extracts were joined and evaporated under reduced pressure to the volume about 100 ml and this concentrate was loaded to a chromatographic column filled with 100 g silica gel (Lichroprep Merck 60, 63-200 μm). The column was washed first with toluene (about 300 ml) and then with a mixture of toluene and from 5 to 30% (v/v) acetone. The fractions containing sirolimus (TLC monitoring) were combined and evaporated to dryness to produce a residue. The

residue (5.5 g) was dissolved in ethyl acetate (20 ml) and 50 ml hexane was added to the solution. The crystallization of sirolimus was accomplished by standing the solution in a refrigerator (about +2° C.) unto crystallization occurred. Crystalline sirolimus was separated by filtration. 2.1 g of crystalline sirolimus was obtained.

### Example 3

10 liter of whole fermentation broth obtained by submerged cultivation of Streptomyces sp. producing tacrolimus was diluted with 10 liter acetone and the suspension was stirred for 2 hours. Then, 2 liter of toluene was added and the mixture was stirred for another 2 hours. Finally the mixture was processed on a centrifuge, obtaining 3.2 liter of the organic extract. The organic extract was further processed as described in the Example 1.

1. A process for isolating macrolide compounds from fermentation broth comprising the steps of:
a. diluting fermentation broth with a water-miscible organic solvent to obtain an aqueous extract,
b. extracting the aqueous extract with a water non miscible solvent at pH from about 5 to about 12 to obtain organic extract,
c. partially evaporating the organic extract to obtain concentrate of macrolide compound,
d. obtaining fractions containing the macrolide compound using chromatography of the concentrate on silica gel using a mixture of toluene and acetone as the mobile phase,
e. concentrating the fractions containing the macrolide compound; and,
f. crystallizing the residue from a suitable solvent.

2. The process of claim 1 wherein the water-miscible organic solvent is selected from the group consisting of ethanol, 1-propanol, 2-propanol or acetone.

3. The process of claim 2 wherein the water-miscible organic solvent is 2-propanol or acetone or a mixture of the 2-propanol and acetone.

4. The process of claim 1 wherein the aqueous extract is separated from the solid phase by filtration or sedimentation prior to subjecting to the treatment with a water non miscible solvent.

5. The process of claim 1 wherein the aqueous extract is not separated from the solid phase before subjecting to the treatment with a water non miscible solvent.

6. The process of claim 1 wherein the water non miscible solvent is selected from the group consisting of toluene, xylene, dichloromethane, dichloroethane, tert-butyl methyl ether or methyl isobutyl ketone.

7. The process of claim 6, wherein the water non miscible solvent is toluene.

8. The process of claim 1 wherein the fermentation broth means whole fermentation broth.

9. The process of claim 6 wherein the whole fermentation broth means a suspension obtained by cultivation of microorganisms Streptomyces sp. that produces a macrolide compound.

10. The process of claim 1 wherein the macrolide compound means tacrolimus or sirolimus or a naturally occurring derivative or analogue of these compounds.

11. The process of claim 1 wherein the concentrate of the macrolide compound means a toluene solution containing a macrolide compound.

12. The process of claim 1 wherein the concentrate is introduced to a column filled with silica gel and said column is washed with toluene stepwise polarized with acetone.

13. The process of claim 12 wherein the volume ratio of toluene and acetone is up to 1:1.

14. The process of claim 1 wherein the fractions containing the macrolide compound are concentrated to a dry residue.

15. The process of claim 1 wherein the suitable solvent used for crystallization of tacrolimus is a mixture of 2-propanol and water.

16. The process of claim 15 wherein the volume ratio of 2-propanol and water is from 1:1 to 1:3.

17. The process of claim 15 wherein the crystallization is accomplished by addition of hexane.

18. The process of claim 17 wherein the quantity of hexane is not limited.

19. The process of claim 1 wherein the suitable solvent for crystallization of tacrolimus or sirolimus is diisopropyl ether or a mixture of ethyl acetate or acetone and hexane.

20. The process of claim 19 wherein the volume ratio of ethyl acetate or acetone and hexane is from 1:1 to 1:5.
